(19) United States
(12) Patent Application Publication
(10) Pub. No.: US 2005/0026999 A1
(43) Pub. Date: Feb. 3, 2005

(54) Composition comprising lactic acid esters of mono-and diglycerides of fatty acids, an emulsifier containing the same and its use

(75) Inventors: Merete Faergemand, Allescod (DK); Flemming Vang Sparso, Skanderborg (DK); Lisbeth Sondberg Svendsen, Arhus (DK); Linda Yvonne Friis, Abyhoj (DK)

Correspondence Address: SCULLY SCOTT MURPHY & PRESSER, PC, 400 GARDEN CITY PLAZA, GARDEN CITY, NY, 11530, US

(73) Assignee: Danisco A/S, Copenhagen K (DK)

(21) Appl. No.: 10/854,978

(22) Filed: May 27, 2004

Related U.S. Application Data
(60) Provisional application No. 60/473,412, filed on May 27, 2003.

(30) Foreign Application Priority Data
May 27, 2003 (GB) 0312147.2

(51) Int. Cl. A61K 31/225

(52) U.S. Cl. 514/547

(57) ABSTRACT

The invention relates to compositions comprising lactic acid esters of mono- and diglycerides of fatty acids i.e. LACTEM compositions. The invention relates especially to novel LACTEM, which include 5 to 95% by weight unsaturated fatty acid(s), as well as to emulsifiers containing LACTEM with unsaturated fatty acids. The use of unsaturated fatty acids in LACTEM improves firmness, volume and/or texture of food and non-food products produced with said LACTEM.

This application claims priority from U.S. Provisional Application No. 60/473,412 filed May 27, 2003.

### FIELD OF THE INVENTION

The present invention relates to a composition comprising lactic acid esters of mono- and diglycerides of fatty acids (said composition hereinafter called LACTEM). It relates especially to LACTEM including unsaturated fatty acid(s) (hereinafter called unsaturated LACTEM). The invention also relates to an emulsifier comprising unsaturated LACTEM and to the use of unsaturated LACTEM to improve firmness, volume and/or texture of products and to food and non-food products containing unsaturated LACTEM.

### BACKGROUND OF THE INVENTION

It is well known in the art that mono- and diglycerides of fatty acids can be used as emulsifiers or surfactants in various products, such as in whipped food and non-food products. It is also known that the free hydroxyl group of the mono- and diglycerides can be esterified with organic acids such as acetic acid, lactic acid, diacetyltartaric acid etc. These esters typically also have emulsifier and surfactant properties. Mono- and diglycerides esterified with lactic acid are called LACTEM and their general nature and production is described by N. Krog (Food Emulsifiers and their chemical and physical properties, in Friberg, S. E. and Larsson, K (Eds.) Food Emulsions, 3^{rd} Ed, Marcel Dekker, New York, p. 150-151, 1997).

According to Krog, LACTEM are based on reactions between lactic acid and monoglycerides based on fully hydrogenated vegetable or animal fats. Such traditional LACTEM (hereinafter also called saturated LACTEM) products are commercially available from Danisco Ingredients, Denmark, e.g. under the trade name Lactodan (old) or GRINDSTED® LACTEM (at present) or as part of blends under the tradename GRINDSTED® PS. The commercial LACTEM have also been described as a mixture of partial lactic acid and fatty acid esters of glycerol.

The use of LACTEM in food products has been described in various patents, such as U.S. Pat. No. 4,826,699, which describes the composition of various Lactodan products based on saturated fats such as palmitic and stearic acid. The patent relates to the production of bakery products, whipped desserts, ice cream, mousse, etc. by treating a conventional emulsifier such as LACTEM with carbon dioxide to improve the foam structure.

U.S. Pat. No. 5,290,581 relates to a whippable non-dairy cream, which may contain Lactodan as an emulsifier. U.S. Pat. No. 5,336,514 relates to a whippable non-dairy cream, which may contain Lactodan as a destabilizing emulsifier in an emulsifier system.

EP 0 807 385 A2 relates to a whey protein based alternative cream which may contain lactic esters of monodiglycerides as emulsifiers. However, there are no examples of any specific LACTEM emulsifiers or of their use.

EP 0 294 119 B1 relates to a liquid artificial cream which may contain lactic esters of monoglycerides as emulsifiers. However, there are no specific examples of any specific LACTEM emulsifiers or of their use.

The old U.S. Pat. Nos. 3,068,103, 3,098,748, 3,351,531, 3,400,722 and 3,443,960 by P. Noznick et al. refer to whipping agents called glycerol lacto oleate or glyceryl lacto monooleate, which according to the specifications function similarly to the saturated stearate or palmitate based products. It is not known how these glycerol or glyceryl lacto products were produced, what their respective proportions of mono- and diglycerides were or what amount of lactic acid was included. There is no indication of how the respective derivatives affected the whippability and the firmness of the whipped product. It should be noted that commercial LACTEM products have always been based on saturated and/or fully hydrogenated fatty acids. The Noznick US patents simply state that the palmitate of glycerol lacto palmitate can be substituted for oleate. However, no examples of the whipping result have been given for the oleate.

It should be noted that all documents cited in this text (“herein cited documents”) as well as each document or reference cited in each of the herein-cited documents, and all manufacturer's literature, specifications, instructions, product data sheets, material data sheets, and the like, as to any product mentioned in this text, are hereby expressly incorporated herein by reference.

Emulsifiers are added to foods for several reasons. They may be used to influence whippability, foam stability and structure, firmness, aeration, cake batter performance and cake volume, overrun, whipping time, spattering, stickiness, viscosity, softness, dough stability, bread staling, etc. Emulsifiers can also be used in plastics to influence the antifog and antistat properties.

Emulsifiers may increase emulsion stability through creation of liquid crystals (as in protein free emulsions) or through cooperative interactions with proteins typically found in food. Another important functionality of emulsifiers is to induce controlled destabilisation. Destabilisation under controlled conditions is very important in whippable emulsions (mousse, whipping creams, ice cream, etc.) and can be achieved by adding emulsifiers of various polarities. The destabilisation process involves several physical changes of the emulsion taking place at low temperature, such as crystallisation of the fat phase and partial desorption of interfacial protein, resulting in a decrease in emulsion stability under shear.

At low temperature emulsifiers such as e.g. monoglycerides displace surface protein partly in ice cream mix and other whippable emulsions. Furthermore the influence of emulsifiers on the visco-elasticity of the interfacial film of the emulsion droplets may be highly important with respect to the partial destabilisation needed in aerated emulsions. Thus, the function of emulsifiers such as monoglycerides and LACTEM in aerated emulsions (e.g. ice cream, whipped cream, mousse, etc.) is to decrease the emulsion stability under shear resulting in formation of clusters of agglomerated fat particles, which during aeration form a structure around and between the air cells, stabilising the foam structure. The agglomerated fat phase contributes to the creaminess and the stability of the foam. In ice cream the melt down stability is directly related to the amount of destabilised fat.

The emulsifiers that typically induce controlled destabilisation are for example monoglycerides, polysorbates, LACTEM and other non-polar emulsifiers. In contrast to the protein displacement by non-ionic emulsifiers (e.g. polysorbates, monoglycerides, LACTEM), a cooperative adsorption of emulsifiers and proteins has been found to take place at O/W interfaces with anionic emulsifiers, such as diacetyl tartaric acid ester of monoglycerides (DATEM).

Typically the destabilisation of the fat phase in the whipped product provides some firmness to the product, through the creation of a network of partially coalesced fat surrounding and connecting the air bubbles. However, usually also gums such as gelatine, xanthan, alginate, guar gum or locust bean gum are used to provide firmness in the whipped product. While stability is a measure of how well the air bubbles stay in the product or how much water leaks out of the whipped product, firmness is a measure of the texture of the final whipped product under pressure. Firmness is an important property in whipped products which are required to have a firm texture. Firmness may also affect the stability by creating a “viscosity barrier” against air bubble coalescence.

Commercially available LACTEM are based on fully hardened fatty acids. They are mainly used in foods such as in aerated desserts, cake shortenings and fats for toppings or in cream imitations. Such saturated LACTEM are often used in combination with saturated monoglycerides for providing a product which is stable in the alfa-crystalline form. Standard saturated LACTEM are known to be good emulsifiers for increasing the whippability of whipped products such as dairy or non-dairy creams, mousses, etc., for improving the aeration and foam stability of topping powders and for improving the aeration and stability of cake batter. However, the prior art saturated LACTEM does not perform especially well in establishing firmness in whipped products.

Thus, there exists a need for providing new LACTEM compositions with new and improved properties. There is especially a need for improving the firmness of the foams produced with LACTEM emulsifiers in order to provide a desired texture, mouthfeel and air bubble stability of whipped products. Firmness, volume and/or texture are especially important in food products such as mousses, whipped dairy and non-dairy creams, ice cream, decoration cream toppings, whipped fillings, sour dairy products and in cake margarines and cake mixes. =dr

### BRIEF DESCRIPTION OF THE INVENTION

The present invention satisfies the need of improved firmness, volume and/or texture in products produced with LACTEM. It solves the problem by substituting a part of the prior art saturated fatty acids with unsaturated fatty acids.

The prior art commercialised LACTEM were all based on fully hardened fatty acids since it was thought that the performance of LACTEM containing unsaturated fatty acids was poor. It has, however, surprisingly been found that replacing a part of the saturated fatty acids with unsaturated fatty acids unexpectedly improves the emulsifier performance of the LACTEM.

According to the invention it has been surprisingly found that incorporation of unsaturated fatty acids in a LACTEM improves the firmness of a foam compared to the use of saturated LACTEM as an emulsifier. Conventional saturated LACTEM are known to provide a good whippability. However, this is generally not the case for LACTEM based on highly unsaturated fatty acids, as will be shown below. It seems probable that the poor whippability provided by highly unsaturated fatty acids in LACTEM has acted as a deterrent on those skilled in the art and this has resulted in that commercial LACTEM are all based on fully hardened fatty acids.

The present invention is based on the realization that a combination of saturated and unsaturated fatty acids in a LACTEM provides whipped products with an excellent firmness while maintaining the whippability at an acceptable and sometimes a very good level. The combination also has been found to improve the volume of whipped products and the texture and mouthfeel of non-whipped food products.

Accordingly, the present invention provides a novel unsaturated LACTEM which is a composition comprising lactic acid esters of mono- and diglycerides of fatty acids, wherein said fatty acids comprise from 95 to 5% by weight unsaturated fatty acid(s) and from 5 to 95% by weight saturated fatty acid(s). Each of said esters contain at least one lactic acid moiety and have the Formula 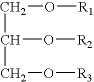
wherein each R₁, R₂ and R₃ independently designates a fatty acid moiety, a lactic acid moiety or a hydrogen atom, provided that at least one of R₁, R₂ and R₃ comprises an unsaturated or saturated fatty acid moiety.

The above Formula is intended to correspond to the formula of the lactic acid esters of standard LACTEM with the difference that at least 5% of the fatty acid moieties derive from unsaturated fatty acids.

The proportions of saturated and unsaturated fatty acids in the unsaturated LACTEM of the present invention may be varied by varying the fats, oils and/or fatty acids used in the production of the LACTEM. It is preferred that the unsaturated LACTEM contains at least 10%, preferably at least 20%, more preferably at least 30% unsaturated fatty acids.

The unsaturated LACTEM of the present invention preferably comprises from 5 to 50%, preferably 10 to 30%, most preferably 13 to 25% by weight of lactic acid moieties, calculated as lactic acid on the total weight of the unsaturated LACTEM after saponification.

The unsaturated LACTEM of the present invention is preferably based on saturated and unsaturated fatty acids which comprise 12 to 24 carbon atoms and the unsaturated fatty acid comprises 1 to 6 double bonds. The preferred unsaturated fatty acids are oleic acid, linoleic acid and linolenic acid

The unsaturated LACTEM of the invention is preferably based on a mono- and diglyceride with a monoglyceride content of from 40 to 99.5%, preferably from 60 to 99%, more preferably from 75 to 97%. The same content of molecules having only one fatty acid moiety may be obtained by producing the LACTEM directly from glycerol, fatty acids and lactic acid.

The present invention also provides an emulsifier containing LACTEM with an effective amount of unsaturated fatty acid moieties, which provides an improved emulsifier performance and improves the firmness, volume and/or texture of products compared to a similar product produced with a saturated LACTEM. The novel emulsifier of the present invention comprises lactic acid esters of mono- and diglycerides of fatty acids, each of said esters containing at least one lactic acid moiety and having the Formula 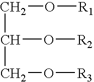
wherein each R₁, R₂ and R₃ independently designates a fatty acid moiety, a lactic acid moiety or a hydrogen atom, provided that at least one of R₁, R₂ and R₃ comprises an unsaturated or saturated fatty acid moiety, said emulsifier comprising an amount of unsaturated fatty acid moieties which is effective in improving the firmness, volme and/or texture of products produced with said emulsifier, especially whipped products.

An effective amount of unsaturated fatty acid moieties has been found to be at least 5% of the fatty acid moieties of the LACTEM. However, the firmness of the foam is greatly improved if the amount of unsaturated fatty acid moieties is increased further to 10 or 20%. The preferred range of unsaturated fatty acid moieties is 55 to 45% by weight of the fatty acid moieties.

It has also been found that the firmness of the whipped product is influenced by the amount of lactic acid moieties in the unsaturated LACTEM. Thus, the unsaturated LACTEM emulsifier of the invention should comprise at least 5%, by weight of lactic acid moieties, calculated as lactic acid on the total weight of the LACTEM after saponification. The firmness is further improved if the amount of lactic acid moieties is increased to 10% or more.

The monoglyceride content of the mono- and diglyceride from which the unsaturated LACTEM is produced also influences its performance as an emulsifier. Thus, it has been found that for best performance of the unsaturated LACTEM, the monoglyceride content of the mono- and diglyceride should be between 40 and 99.5%, preferably between 60 and 99%, more preferably between 75 and 97%. The same content of molecules having only one fatty acid moiety may be obtained by producing the LACTEM directly from glycerol, fatty acids and lactic acid.

The present invention also relates to the use of the unsaturated LACTEM as an emulsifier for improving the firmness, volume and/or texture of emulsified products, especially whipped products. The preferred products are food products such as protein and/or fat containing food products, but the invention is applicable also for improving the firmness of non-food whipped products.

Finally, the present invention relates to food and non-food produces which contain an effective firmness, volume and/or texture improving amount of the unsaturated LACTEM of the present invention. Preferred food products are mousses, whipped dairy and non-dairy creams (e.g. vegetable fat whipping creams or so called imitation dairy creams), decoration creams, toppings, ice creams, sour dairy products such as yogurts whipped fillings and cake margarines and/or mixes.

### DETAILED DESCRIPTION OF THE INVENTION

In the present specification and claims the term LACTEM means a composition comprising lactic acid esters of mono- and/or diglycerides of fatty acids. The traditional LACTEM based on fully hardened fats is called “saturated LACTEM” and the LACTEM of the present invention, wherein 95 to 5% by weight of the fatty acids are unsaturated fatty acids and 5 to 95% by weight are saturated fatty acids, is called “unsaturated LACTEM”. The LACTEM composition is typically a mixture of esterified and non-esterified glycerides, wherein, in this connection the “esterified” refers to esters with lactic acid and the “glyceride” refers to esters with fatty acids. Thus, a typical LACTEM contains also a significant amount of non-esterified (non-lactylated) mono- and diglycerides. The term “comprising” used in the specification is further intended to indicate that the composition may contain also other components than the lactic acid esters of the Formula shown above. Thus, in addition to the mono- and diglycerides an industrially produced LACTEM may contain minor amounts of free glycerol, free fatty acids, triglycerides of fatty acids, etc.

The amounts of fatty acid-moieties and lactic acid moieties in the LACTEM referred to in the present specification and claims are calculated on an analysis made after the LACTEM has been chemically reacted, since it is not possible to define the different acids in the LACTEM as such. The distribution of the fatty acid moieties is calculated on the total weight of fatty acids in the LACTEM, while the amount of lactic acid moieties is calculated on the total weight of the LACTEM. The fatty acid distribution may be calculated by interesterification of the LACTEM with methanol and by analysing the methyl esters of the fatty acids by gas chromatography. The lactic acid amount may be analysed by saponifying the LACTEM and analysing the amount of lactic acid derivatives present.

In the present specification and claims the term “firmness” of a whipped product refers to the texture of the foam under pressure. The firmness of a whipped product is measured as the maximum force measured during compression (peak force). The unit of the firmness is gram (g). The “improved firmness”, discussed in the specification and claims, refers to the improved firmness obtainable with the novel unsaturated LACTEM as compared to the firmness obtainable under similar conditions with traditional saturated LACTEM.

The unsaturated LACTEM of the present invention can be produced by causing reaction between lactic acid and mono- and/or diglycerides of fatty acids wherein at least 5% of the fatty acids are unsaturated fatty acids. Alternatively, glycerol may be directly esterified with lactic acid and desired unsaturated and saturated fatty acids. The amount of lactic acid and fatty acids, respectively, may be controlled by providing the reagents in a desired molar ratio. The reaction conditions also influence the distribution of the various components in the LACTEM.

In the reaction between glycerol and natural fats, which mainly consist of triglycerides, the reaction product includes triglycerides, 1,3-diglycerides, 1,2-diglycerides, 1-monoglycerides and 2-monoglycerides and glycerol. The proportion between components of the reaction product is controlled by the molar ration between the raw materials. The glycerol is preferably removed by distillation prior to the esterification with lactic acid. After removal of the glycerol the reaction mixture maybe used directly for the esterification with lactic acid. The triglycerides cannot be esterified with lactic acid since they lack a free hydroxyl. The triglycerides may be removed by distilling off the mono- and/or diglycerides. The proportion of mono and diglycerides may be adjusted by controlling the composition of the distillate e.g. by fractional distillation(s), or by controlling the molar ration between the glycerol and triglyceride raw materials in the reaction mixture.

An effective method for preparing unsaturated LACTEM is to react unsaturated mono- and/or diglycerides with lactic acid. Usually reaction temperatures of 110 to 250° C., pressures of 0.5 to 200 kPa and reaction times from about 1 to 6 hours are employed. Water soluble bitter and/or sour tasting components such as free lactic acid and glycerol mono-, di- and/or poly-lactin can subsequently be removed by washing with water.

Unsaturated LACTEM can also be prepared by reacting glycerol, unsaturated fatty acids or unsaturated triglycerides with lactic acid with or without a catalyst, usually at about 110 to 250° C., pressures of 0.5 to 200 kPa and reaction times of about 1 to 6 hours with subsequent purifying by washing. The composition of the unsaturated LACTEM is controlled by the molar ratio between the glycerol, unsaturated fatty acids and/or triglycerides and the lactic acid.

Irrespective of which production method is used, the unsaturated LACTEM comprises a mixture of mono- and/or diglycerides of fatty acids and lactic acid esters of mono- and diglycerides of fatty acids. The lactic acid esters contain esterified lactic acid distributed on a number of isomeric compounds, as is known in the art. Unsaturated mono- and/or diglycerides are prepared by methods know to the man skilled in the art. E.g. a mixture of mono- and diglycerides can be prepared by reacting glycerol and unsaturated triglycerides or unsaturated fatty acids in the presence of an alkaline catalyst. Eventually, the components can be concentrated and purified by thin film high vacuum distillation or short path distillation in order to prepare mixtures of distilled mono- and diglycerides especially suitable for the preparation of unsaturated LACTEM.

The preferred glyceride for producing unsaturated LACTEM is the 1-monoglyceride. Although the emulsifier properties of the unsaturated LACTEM of the present invention are surprisingly not very significantly influenced by the monoglyceride content of the mono- and diglyceride from which the unsaturated LACTEM is produced, it has been found to be preferable to have a monoglyceride content of from 40 to 99.5%, preferably from 60 to 99%, more preferably from 75 to 97% in the mono- and diglyceride mixture used for the preparation of the unsaturated LACTEM.

The lactic acid will react with a free hydroxyl in the 1, 2 and/or 3-position. Sterically the most likely position for lactic acid in the 1-monoglyceride is the 3-position. Lactylation has been found to have a very significant influence on the texture and air distribution in foams produced with the present unsaturated LACTEM as an emulsifier. Thus, lactylation of monoglycerides and mono- and diglycerides provides a firmer foam with a finer air distribution than in foams obtained with the corresponding unlactylated monoglycerides and mono- and diglycerides.

The degree of lactylation has also been found to influence the firmness of the foam obtained. Thus, the monoglycerides or mono- and diglycerides should be lactylated with at least 5% of lactic acid. The unsaturated LACTEM should most preferably contain from 13 to 25% by weight of lactic acid moieties, calculated as lactic acid on the total weight of the LACTEM after saponification.

The influence of lactylation has not been found to increase linearly with the level of lactylation and the difference in functionality was found to be small when the level of lactylation was increased from 13 to 22%.

The unsaturated LACTEM of the present invention is a composition based on both saturated and unsaturated fatty acids and both mono- and diglycerides. Some but not necessarily all individual glycerides are lactylated. The lactylated glycerides are lactic acid esters containing at least one lactic acid moiety and they may be represented by the Formula 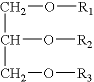
wherein each R₁, R₂ and R₃ independently designates a fatty acid moiety, a lactic acid moiety or a hydrogen atom, provided that at least one of R₁, R₂ and R₃ comprises an unsaturated or saturated fatty acid moiety. The lactic acid moieties may comprise a single lactic acid ester or it may be a chain of lactic acid esters. Typically such a chain is an oligomer chain of 2 to 5 lactic acid units. The fatty acid moiety may be attached to the glycerol backbone or to the lactic acid moiety.

The fatty acids which are used in the production of the unsaturated LACTEM of the present invention preferably comprise 12 to 24 carbon atoms, and more preferably 16 to 22 carbon atoms, most preferably 16 to 18 carbon atoms. The unsaturated fatty acid preferably comprises 1 to 6 double bonds.

The unsaturated fatty acids are preferably selected from myristoleic acid, palmitoleic acid, oleic acid, eliadeic acid, linoleic acid, linolenic acid, octadecatetraenoic acid, gadoleic acid, eicosadienoic acid, arachidonic acid, eicosapentenoic acid, erucic acid, docosapentenoic acid and docosahexaenoic acid. The unsaturated fatty acid preferably has 18 carbon atoms and it is preferred that at least 50% by weight of the unsaturated fatty acids is selected from oleic acid, linoleic acid and linolenic acid.

The fatty acid of the unsaturated LACTEM of the invention is preferably derived from oils and/or fats of vegetable and/or animal origin. Suitable oils and fats are selected from almond oil, babassu oil, butter oil, chicken fat, cocoa butter, coconut oil, cotton seed oil, fish oil, hazelnut oil, illipe fat, kokorm, lard, linseed oil, maize oil, mango, menhaden oil, olive oil, palm kernel oil, palm oil, peanut oil, rapeseed oil (high and low erucic), safflower oil, high oleic safflower oil, seal oil, shea fat, single cell algae oil, soybean oil, sunflower oil, high oleic sunflower oil, tall oil, tallow and a mixture of any of these oils and/or fats.

The degree of unsaturation of fats and oils may be indicated by the iodine value (IV), which is defined as grams of added iodine to the fatty acid per gram of sample. The fatty acids included in the unsaturated LACTEM of the present invention may be derived from oils and/or fats having an iodine number between 6 and 160, preferably between 25 and 120, more preferably between 50 and 100, most preferably between 85 and 95. Correspondingly, the unsaturated LACTEM of the present invention should have an iodine value above 6 and preferably between 10 and 120. It is more preferable that the LACTEM has an iodine value of 20 to 100 and most preferable that the iodine value is from 35 to 75 to provide a proper balance between unsaturated and saturated fatty acids. It should be noted that the traditional LACTEM based on fully hydrogenated fatty acids may have an iodine value of 2 to 3 and up to about 5 due to incomplete hydrogenation of the fats and/or oils or fatty acids used for the production of the traditional LACTEM. However, an iodine value of 5 or less indicates a too low amount of unsaturated fatty acids and has not been found to provide the desired improved firmness in the foam.

Since the degree of unsaturation in the fatty acids of the unsaturated LACTEM of the present invention affects the emulsifier properties of the LACTEM, the content of unsaturated fatty acid in any selected oil and/or fat may be influenced by partial and/or selective hydrogenation of the unsaturated fatty acids contained in said oil and/or fat. The hydrogenation should preferably be performed prior to reaction of said oil and/or fat with glycerol.

The unsaturated LACTEM of the present invention may be used to improve the firmness of non-food and food products. The non-food products include cosmetical products (e.g. creams, lotions, hair products etc.) and pharmaceutical products. The food products include whipped foods which preferably contain fats and/or proteins. The whipped products should contain an effective amount of the unsaturated LACTEM for improving the firmness of the whipped product. An effective amount of the unsaturated LACTEM is one which significantly increases the firmness of the whipped product compared to the same product whipped with a standard saturated LACTEM.

The amount of unsaturated fatty acids should be at least 5% and preferably more than 10% to increase the firmness compared to saturated LACTEM. Very good results have been obtained with emulsifiers where the LACTEM contained 55 to 45% by weight unsaturated fatty acid moieties and from 45 to 55% by weight saturated fatty acid moieties. The preferred fatty acids are those having 18 carbon atoms and 1, 2 or 3 double bonds, i.e. oleic acid, linoleic acid and linolenic acid.

For improving the firmness of the foam, the level of lactic acid moieties in the emulsifier system should be from 5 to 50%, preferably 10 to 30%, most preferably 13 to 25% by weight, calculated as lactic acid on the total weight of said LACTEM after saponification.

The whippd food product emulsified with the unsaturated LACTEM of the present invention preferably contains fat and or protein to assist in providing a firm foam.

The proteins included in the protein containing food products emulsified with the use of the present unsaturated LACTEM are preferably selected from milk protein, caseinate, whey protein, soy protein, pea protein, oat protein, potato protein, cottonseed protein and mixtures thereof. The protein interacts with the fat and emulsifier system and improves the viscosity of the mixture to be aerated. Protein also increases whippability.

The fats included in the products emulsified according to the invention include dairy and non-dairy fats. The non-dairy fats include partially or fully hydrogenated vegetable fats from e.g. coconut, palm, palm kernel. A large amount of fat, such as in a cake margarine, seems to improve the whippability of the unsaturated LACTEM of the present invention.

The food products most typically whipped with the present unsaturated LACTEM are mousses, whipped dairy creams, decoration creams, toppings, vegetable fat based creams (imitation creams), ice creams, whipped fillings, cake margarines and cake mixes. The products may be high fat, low fat or even non-fat products, e.g. high fat mousses, low fat mousses and fat free mousses. Mousses, ice creams and cake margarines can be whipped with the unsaturated LACTEM as the only emulsifier. The natural and artificial creams and toppings are preferably whipped with an emulsifier system which in addition to the unsaturated LACTEM contains another emulsifier which acts to stabilize the emulsion from flocculation and coalescence during storage (before whipping), while the unsaturated LACTEM provides the required destabilization of the emulsion under shear. In cake mixes it is also preferred to use other emulsifiers in addition the unsaturated LACTEM. Sour dairy products such as yogurts show improved texture and reduced grittiness when emulsified with the unsaturated LACTEM of the present invention.

The present invention is illustrated in the following by some examples.

### EXAMPLE 1

Production of Unsaturated LACTEM

1100 grams of distilled mono- and diglyceride based on sunflower oil (IV of the distilled mono- and diglyceride=111) and having a monoglyceride concentration of 95% by weight, the rest being a mixture of diglyceride and minor components, was reacted with 300 grams of a 88% by weight lactic acid in water in a 2 L three necked reaction flask with mechanical agitation. The reaction was carried out at up to 210° C. and a pressure of 5 kPa for 3 hours. The resulting reaction mixture was washed with two times 511 grams of water at 90° C. To remove traces of bitter components the washed product was deodorized with live steam at 160° C. and a pressure of 10 Pa for 30 min.

The resulting unsaturated LACTEM was saponified and was found to contain 89% unsaturated fatty acids and 11% saturated fatty acids based on the weight of fatty acids. It was also found to contain 15% lactic acid based on the total weight of the LACTEM after saponification.

### EXAMPLE 2

The Firmness of Mousses

Mousses were made according to a standard recipe.
Cream, 38% 19.57%
Skim milk 63.72%
Sucrose 14.5%
Gelatine 3250 1.6%
Emulsifier 0.6%
Nisin 0.01%
   Process
1. Mix liquid ingredients
2. Mix dry ingredients
3. Mix dry and liquid ingredients with constant agitation at 70° C. MINI UHT heating
4. Homogenisation at 75° C./150 bar
5. Pasteurisation at 78° C./2 min
6. Cooling to 10° C.
7. Ageing at 5° C. for 1 hour
8. Mixing on Mondo mixer: Parameters:
9. Filling, 8 beakers (155 mm height)

Furthermore, some mousse mixes were—instead of mixing to constant overrun in the Mondo mixer in pilot scale—mixed manually on a Hobart mixer in the laboratory. The mousse mixes were aged for 1 hour at 5° C. and then whipping was performed at speed 3 and maximum overrun was measured; this was called whippability. The experiments were performed in duplicate.

The tested LACTEM were a standard saturated LACTEM based on hardened palm oil, an unsaturated LACTEM based on highly unsaturated sunflower oil (the degree of unsaturation of the fatty acids was about 89%) and a medium unsaturated LACTEM based on a mixture of palm oil and sunflower oil (the degree of unsaturation was 45%). The firmness of the mousses was as follows:

Firmness and consistence was measured in the mousse beakers by Texture Analyser (TA instruments) using a 35 mm back extrusion rig (A/BE35) with the following parameter settings:
TA Mode: Measure Force in Compression
TA Option: Return to start
Pre-speed: 1.0 mm/s
Speed: 1.0 mm/s
Post-speed: 1.0 mm/s
Distance: 20 mm
Trigger: 10 g

Firmness is defined as the maximum force measured during compression (peak force).

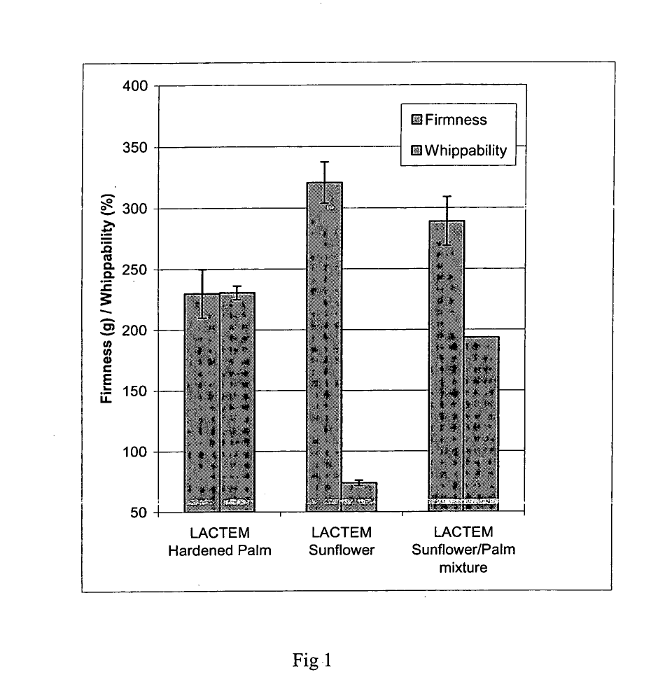

The results are shown in the graph of FIG. 1 and indicate that whereas the standard LACTEM made from saturated fat gives rather low firmness, it provides fine whippability. The highly unsaturated LACTEM provided poorer whippability at constant whipping conditions applied in the Hobart mixer but improved the firmness. However, the medium unsaturated LACTEM based on both saturated and unsaturated fatty acids provided both an improved firmness and good whippability.

### EXAMPLE 3

Low Fat Mousse

Low fat mousses are made according to the recipe below

Low fat yoghurt mousse formulation:

22.55% Skimmed milk
4.00% Skimmed milk powder
60.00% Yogurt natural
11.00% Sucrose
1.85% Stabiliser blend (gelatine/starch)
0.60% Unsaturated LACTEM
   + Flavours
   + Colours
100.00% Total
   Processing procedure:

The mousse is based on a cream mix and yoghurt.

Cream mix:

1. Heat all liquid ingredients to 40° C.
2. Add dry ingredients
4. Homogenise at 200 bar at 65° C.
3. Pasteurise at 95° C. for minutes
5. Cool to 10° C.
6. Add 60% yogurt to 40% mix
7. Age for minimum 30 minutes
8. Whip in aerator to 80% overrun
9. Fill

The firmness of the low fat mousse is almost as good as that of the high fat mousse of example 2.

### EXAMPLE 4

The Firmness of Whipped Decoration Creams

A decoration/chantilly cream was made according to the formulation and process below:

Formulation (per cent) %

Cream (38% fat) 71.00
Skimmed milk 19.50
Sugar 8.00
LACTEM* 0.525
Calatine (3250F.) 0.975
Total 100.00
*The type of LACTEM was varied to compare the different LACTEMs
   Process
1. Mix dry ingredients into the standardised cream
2. Pre-heat to 75° C.
3. Upstream homogenisation at 150 bar
4. UHT treat at 142° C. for 3-4 seconds
5. Cool to 10° C. or below
6. Ageing for at least 1 hour
7. Whip in an aerator to desired overrun (180-200%)
8. Store at 5° C.

Creams were prepared with a standard saturated LACTEM; GRINDSTED® LACTEM P22 and an experimental unsaturated LACTEM based on sunflower oil (LACTEM 2304/38), respectively. Sunflower contains 89% unsaturated fatty acids, predominantly linoleic acid

The creams were whipped on a Mondomixer to a constant overrun of 180%, and the firmness of the foams was measured using a TA XT2 Texture Analyser (Stable Microsystems). The firmness of the different foams can be seen in Table 1.

TABLE 1
Foam firmness of decoration
creams prepared with different LACTEM

| Emulsifier | Foam Firmness (g) |
|---|---|
| GRINDSTED ® LACTEM P22 | 8.6 ± 2.0 |
| LACTEM 2304/38 | 25.8 ± 8.0 |
| Sunflower oil | |

It is clear from Table 1 that use of the new unsaturated LACTEM based on sunflower oil results in much firmer decoration cream, which will be able to keep its shape once put on the subject (e.g. cake, dessert, ice cream) that should be decorated.

### EXAMPLE 5

The Firmness of Whipped Non-dairy Creams

A vegetable fat (hardened palm kernel oil) whipping cream (imitation dairy cream) was made according to the recipe and process below:

Formulation (per cent) %

Vegetable fat (HPKO) 28.00
Skimmed milk 5.50
Na-caseinate 0.66
Sucrose 8.00
Sorbitol, 70% 1.00
GRINDSTED® WP 920 (stabilizer) 0.45
LACTEM* 0.6*
PANODAN® 165 DATEM 0.25
Flavouring 0.2
Demineralised water 55.34
Total 100.00
*The type of LACTEM was varied.
   Process
1. Melt fat and LACTEM
2. Add the rest of the ingredients to cold water
3. Heat water solution to 70° C.
4. Add fat phase to the water phase at 70° C.
5. UHT treatment at 142° C./3 seconds
6. Homogenise at 150 bar/75° C.
7. Cool to 5° C.
8. Fill
9. Store cold at 5° C. for 24 hours

Creams were prepared with either 0.6% of a standard saturated LACTEM; GRINDSTED® LACTEM P22 or an experimental unsaturated LACTEM based on sunflower oil (LACTEM 2304/38), respectively. The new unsaturated LACTEM (LACTEM 2304/38) was tested in two different dosages: 0.3% and 0.6%, respectively.

After storage for 24 hours at 5° C. the vegetable creams were whipped on a Hobart mixer for 180 s, and the resultant overrun was measured. Furthermore the foam firmness was measured using a Boucher Electronic Jelly Tester (S. Gardia Ltd.). The results for the individual creams are given in Table 2 in arbitrary units.

When using the new unsaturated LACTEM 2304/38 in the same dosage as the standard saturated GRINDSTED® LACTEM P22 the vegetable cream gelled in the flask, due to too strong destabilisation. Thus, no whipping test could be performed with this dosage of the unsaturated LACTEM. In a dosage of 0.3%—i.e. half of that used for the standard saturated LACTEM—the unsaturated LACTEM (2304/38) performed equally well as the standard saturated LACTEM, as shown in Table 2. It can be seen that the foam firmness was improved from 64 to 84, with a slight decrease in overrun.

However, with the new unsaturated LACTEM (2304/38) it was observed that the whipping time could also be reduced. The same foam firmness could be obtained with the unsaturated LACTEM (2304/38) in 150 seconds as with the standard saturated GRINDSTED® LACTEM P22 in 180 seconds whipping time.

TABLE 2
Properties of vegetable creams made with different LACTEMs.

| | | | Foam |
|---|---|---|---|
| Emulsifier | Dosage (%) | Foam overrun (%) | firmness |
| GRINDSTED ® | 0.6% | 235 | 64 |
| LACTEM P22 | | | |
| LACTEM 2304/38 | 0.6% | Sample had gelled in | — |
| Sunflower oil | | bottle - No whipping | |
| | | possible | |
| LACTEM 2304/38 | 0.3% | 201 | 84 |
| Sunflower oil | | | |
| LACTEM 2304/38 | 0.3%* | 205* | 66* |
| Sunflower oil | | | |

*In this experiment whipping time was decreased to 150 s.

### EXAMPLE 6

The Firmness of Ice Creams

Ice cream was produced according to the recipe and process outlined below. Two types of LACTEM were compared in the ice cream recipe: a LACTEM based on fully hardened palm oil (“Saturated LACTEM”) and one made from sunflower oil (“Unsaturated LACTEM”).

Ice Cream Recipe in Percent (%)

Water 63.81
Hardened coconut oil, MP 31° C. 8.00
Skimmed milk powder 9.00
Traditional whey powder 2.50
Sucrose 12.00
Glucose Syrup powder 32 DE 4.00
LACTEM* 0.30
LBG 246 0.10
Guar Gum 1400 0.10
Carrageenan 1035 0.02
Vanilla Flavouring 3134, NI 0.10
Colouring 0.07
Total 100.00
Total Fat 8.10
Total MSNF 10.95
Total Dry Matter 35.55

* The type of LACTEM was varied as described in the text. The LACTEM were compared to the standard product used in ice cream Cremodan® Super, which is a monodiglyceride emulsifier.

Process

1. Melt the fat at approximately 50° C.
2. Mix liquid ingredients at 20-22° C.
3. Mix dry ingredients
4. Add flavouring
5. Add colouring
6. Add the fat and increase temperature to 65° C.
7. Homogenise at: 78° C./180 bar
8. Pasteurise at: 84° C./30 seconds
9. Cool to 5° C.
10. Ageing overnight in ice water
11. Measure viscosity
12. Freezing, drawing temperature: −5° C. with 100% overrun
13. Fill
14. Overnight freezing in hardening tunnel at −30° C.
15. Store at −25° C.

The melt down resistance was measured by the following method: A rectangular piece of ice cream (125 cc, dimension: approx. 100 mm×50 mm×25 mm) is weighed and placed on wire netting. The room, in which the melting process takes place, is kept at a constant temperature of 22C.+/−1C. The wire netting is placed above a 500 ml glass beaker placed on an analytical balance. The continuous drip of melted ice cream through the net and into the beaker is monitored be increasing weight on the balance.

| | Percentage ice cream |
|---|---|
| Emulsifier | melted after 120 min (%) |
| “Saturated LACTEM” | 33% |
| “Unsaturated LACTEM” | 16% |
| Cremodan Super | 12% |

It is clear that ice cream made with “Saturated LACTEM” has a poor melt down stability, as 33% of the ice cream has dripped through the net after 120 min. The “Unsaturated LACTEM”, however, results in much less melting of the ice cream, and the level of melted ice cream after 120 min is comparable to when using Cremodan Super. The improved meltdown properties of the ice cream, when using “Unsaturated LACTEM” compared to when using “Saturated LACTEM” indicates that an ice cream made with unsaturated LACTEM will show better form stability, due to increased destailisation of the fat phase and thus higher firmness of the aerated ice cream (at least before freezing) than the ice cream made with saturated LACTEM.

### EXAMPLE 7

Cake Mix

Batches of 3000 g cake margarine was produced according to the recipe shown below.

Trial no.

| | 1 | 2 |
|---|---|---|
| WATER PHASE: | | |
| Water | 18.9 | 18.9 |
| Salt | 1 | 1 |
| Potassium sorbate | 0.1 | 0.1 |
| EDTA | 0.015 | 0.015 |
| WATER PHASE total | 20 | 20 |
| FAT PHASE: | | |
| Coconut 31 | 25 | 25 |
| Soya 41 | 18 | 18 |
| Soya 35 | 25 | 25 |
| Rape seed oil | 32 | 32 |
| Palm oil | | |
| FAT IN PARTS total | 100 | 100 |
| FAT total | 78.5 | 78.5 |
| GRINDSTED ® LACTEM F 15 | 1 | |
| LACTEM 2304/38 | | 1 |
| DIMODAN ® HP | 0.5 | 0.5 |
| PPM β-carotene | 3 | 3 |
| FAT PHASE total | 80 | 80 |
| RECIPE total | 100 | 100 |
| FLAVORING no. (w or. o) | | |
| Butter 2722o | 0.02 | 0.02 |

The following process was followed:
1. The water phase ingredients were blended. The water phase was pasteurised by heating to 80° C. and cooling to 50-55° C. pH was adjusted to 5.5.
2. The fat blend was melted and blended, and tempered to 50-55° C. Beta carotene was added.
3. Heat the LACTEM and DIMODAN® HP with some of the fat blend in a ratio of 1:5 to a temperature (75-80° C.) which is 5-10° C. higher than the melting point of the DIMODAN® HP. When this blend is completely melted and well stirred, add it to the remaining heated fat blend, stirring continuously. In experiment 1 GRINDSTED® LACTEM F 15 was used; in experiment 2 the experimental unsaturated LACTEM was used (LACTEM 2304/38).
4. Add the flavouring.
5. Make the emulsion by adding the water phase to the fat phase, stirring vigorously.
6. Crystallize and knead in a tube chiller (NH₃−15 to −20° C. and normal capacity).

Pound cakes were produced with the experimental margarines according to the procedure below:

Recipe:

| Ingredients | % | g |
|---|---|---|
| Sugar 35/20 | 24.9 | 250 |
| Cake flour, Albatros | 12.4 | 125 |
| Wheat starch | 12.4 | 125 |
| Baking powder | 0.5 | 5.0 |
| Past. Liq. Whole egg | 24.9 | 250 |
| Cake margarine | 24.9 | 250 |

Equipment:
Mixer: Hobart N50 with a paddle
Oven: Simon cake oven
   Procedure:

All ingredients must be tempered to room temperature.
1. Mix all ingredients for 1 min. at 1^{st} speed—scrape down
2. Mix for 1 min. at 2^{nd} speed—scrape down
3. Mix for 2 min. at 3^{rd} speed
4. Measure the volume of the batter
5. The poundcake tins are sprayed with “Babette” oil spread, and covered with paper
6. Scale 2×350 g into the pound cake tins
7. Spread out the mass evenly with a spatula
8. Bake for 50 min. at 180° C.
9. After baking take the tins out of the oven, and “drop” it on the table, before taking the cakes out of the tins
10. Take paper off the cakes and turn the right side up
11. The cakes are cooled on a grating for 40 min. before weighing and measuring of the volume

Cake specific volume was measured using rape seed displacement. The results are shown below:

| | | Cake specific |
|---|---|---|
| LACTEM sample | Replicate | volume (ml/g) |
| GRINDSTED ® LACTEM F 15 | 1 | 2.99 |
| LACTEM 2304/38 | 1 | 3.16 |
| GRINDSTED ® LACTEM F 15 | 2 | 2.90 |
| LACTEM 2304/38 | 2 | 3.26 |

The new unsaturated LACTEM, Lactem 2304/38 improved the emulsifier performance and dough stability of the pound cake doughs and resulted in significantly increased cake volumes of the pound cakes. The volume increase was approximately 9%.

### EXAMPLE 8

Low-fat Yogurt

The unsaturated LACTEM of the present invention was tested in a standard yogurt recipe even though LACTEM is not conventionally used in yogurt. A low-fat yogurt was produced according to the following recipe:

| | Dosage (%) |
|---|---|
| Ion exchanged water | 88.73 |
| Skimmed milk powder | 11.05 |
| Pectin LC 1700 | 0.1 |
| Emulsifier/fat | 0.1 |
| YO-MIX ™ 301 | 0.02 u |

Process:
1. Mix all powder ingredients and add the dry blend to the milk/water under good agitation at 45° C.
   Mini UHT
2. Pre-heat to 65° C.
3. Homogenise at 65° C./200 bar
4. Pasteurise at 95° C. for 6 minutes
5. Cool to 5° C.
6. Add starter culture, YO-MIX™ 301 (supplied by Danisco) in a dosage of 0.02 unit/l
7. Fermentation to pH 4.60
8. Cool on plate heat exchanger to 24° C.
9. Fill

Samples were produced with the following emulsifiers:

| | | | Fermentation |
|---|---|---|---|
| | | Dosage | time up to |
| Sample | Emulsifier/fat | (%) | pH 4.6 (hours) |
| 1 | — | — | 7.5 |
| 2 | POLAWAR 70 | 0.1 | 7.5 |
| 3 | GRINDSTED ® LACTEM R110 | 0.1 | 7.5 |

POLAWAR 70 is a hydrogenated and deodorized palm kern oil (supplied by Aarhus United A/S, Denmark); GRINDSTED® LACTEM R 110 is an unsaturated LACTEM according to the present invention and based on selectively hardened rapeseed oil (supplied by Danisco).

Sample 1 was a reference with no emulsifier added. Sample 2 was another reference with fat added, in order to evaluate if any detected effect arises from the emulsifier or whether the effect is a pure fat-derived effect. Sample 3 was produced with unsaturated LACTEM as emulsifier.

The samples were evaluated by an expert panel after 2 and 21 days (3 weeks) of production.

From a sensory point of view samples 1 and 2 were very similar. They were both somewhat grainy/gritty and thus it can be concluded that any effect is an emulsifier effect and not a pure fat effect. The sample with the unsaturated LACTEM of the present invention was clearly smoother than the reference samples.

The results show that graininess/grittiness in low-fat yogurt with pectin added prior to fermentation is reduced by adding unsaturated LACTEM as an emulsifier.

Unsaturated LACTEM improves the texture and mouthfeel of yoghurt made with pectin. Yogurt produced with unsaturated LACTEM was found to have an acceptable viscosity, good smoothness and creaminess.

1. A composition comprising lactic acid esters of mono- and diglycerides of fatty acids (LACTEM), wherein said fatty acids comprise from 95 to 5% by weight unsaturated fatty acid(s) and from 5 to 95% by weight saturated fatty acid(s), each of said esters containing at least one lactic acid moiety and having the Formula 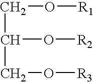
wherein each R₁, R₂ and R₃ independently designates a fatty acid moiety, a lactic acid moiety or a hydrogen atom, provided that at least one of R₁, R₂ and R₃ comprises an unsaturated or saturated fatty acid moiety.

2. The LACTEM according to claim 1, wherein at least 10%, preferably at least 20%, more preferably at least 30% of said fatty acids comprise unsaturated fatty acid(s).

3. The LACTEM according to claim 1, wherein 55 to 45% by weight of said fatty acids comprise unsaturated fatty acid(s).

4. The LACTEM according to claim 1, which is a mixture of mono- and/or diglycerides of fatty acids and said lactic acid esters of mono- and diglycerides of fatty acids.

5. The LACTEM according to claim 4, which comprises from 5 to 50%, preferably 10 to 30%, most preferably 13 to 25% by weight of lactic acid moieties, calculated as lactic acid on the total weight of said LACTEM after saponification, each lactic acid moiety comprising a single lactic acid ester or a chain of lactic acid esters.

6. The LACTEM according to claim 1, wherein said fatty acids comprise 12 to 24 carbon atoms and wherein said unsaturated fatty acids comprise 1 to 6 double bonds.

7. The LACTEM according to claim 6, wherein said unsaturated fatty acid(s) is/are selected from the group consisting of acids selected from the group consisting of myristoleic acid, palmitoleic acid, oleic acid, eliadeic acid, linoleic acid, linolenic acid, octadecatetraenoic acid, gadoleic acid, eicosadienoic acid, arachidonic acid, eicosapentenoic acid, erucic acid, docosapentenoic acid and docosahexaenoic acid.

8. The LACTEM according to claim 7, wherein at least 50% by weight of said unsaturated fatty acid is selected from oleic acid, linoleic acid and linolenic acid

9. The LACTEM according to claim 1, wherein said fatty acids are derived from oils and/or fats of vegetable and/or animal origin.

10. The LACTEM according to claim 9, wherein said oil or fat is selected from the group consisting of almond oil, babassu oil, butter oil, chicken fat, cocoa butter, coconut oil, cotton seed oil, fish oil, hazelnut oil, illipe fat, kokorm, lard, linseed oil, maize oil, mango, menhaden oil, olive oil, palm kernel oil, palm oil, peanut oil, rapeseed oil (high and low erucic), safflower oil, high oleic safflower oil, seal oil, shea fat, single cell algae oil, soybean oil, sunflower oil, high oleic sunflower oil, tall oil, tallow and a mixture of any of these oils and/or fats.

11. The LACTEM according to claim 10, wherein the content of unsaturated fatty acid in said oil and/or fat has been reduced by partial and/or selective hydrogenation of unsaturated fatty acids.

12. The LACTEM according to claim 1, having an iodine value above 6, preferably between 10 and 120, more preferably between 20 and 100, most preferably between 35 and 75.

13. The LACTEM according to claim 1, wherein said monoglycerides comprise from 40 to 99.5%, preferably from 60 to 99%, more preferably from 75 to 97% of said mono- and diglycerides.

14. An emulsifier containing a composition comprising lactic acid esters of mono- and diglycerides of fatty acids (LACTEM) each of said esters containing at least one lactic acid moiety and having the Formula 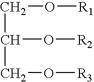
wherein each R₁, R₂ and R₃ independently designates a fatty acid moiety, a lactic acid moiety or a hydrogen atom, provided that at least one of R₁, R₂ and R₃ comprising an unsaturated or saturated fatty acid moiety, said emulsifier comprising an amount of unsaturated fatty acid moieties which is effective in improving the firmness, volume and/or texture of products produced with said emulsifier.

15. The emulsifier according to claim 14, wherein said amount of unsaturated fatty acid moieties is effective in improving the firmness of products whipped with said emulsifier.

16. The emulsifier according to claim 14, wherein said effective amount of unsaturated fatty acid moieties comprises at least 5%, preferably at least 10%, more preferably at least 20%, most preferably at least 30% by weight of the fatty acid moieties of said LACTEM.

17. The emulsifier according to claim 16, wherein said LACTEM comprises 55 to 45% by weight unsaturated fatty acid moieties and from 45 to 55% by weight saturated fatty acid moieties.

18. The emulsifier according to claim 14, wherein said LACTEM also contains non-esterified glycerides of fatty acids and comprises from 5 to 50%, preferably 10 to 30%, most preferably 13 to 25% by weight of lactic acid moieties, calculated as lactic acid on the total weight of said LACTEM after saponification.

19. The emulsifier according to claim 14, wherein said fatty acid moieties comprise 12 to 24 carbon atoms and said unsaturated fatty acid moieties comprise 1 to 6 double bonds.

20. The emulsifier according to claim 19, wherein said unsaturated fatty acid moieties are based on fatty acids selected from the group consisting of acids selected from the group consisting of myristoleic acid, palmitoleic acid, oleic acid, eliadeic acid, linoleic acid, linolenic acid, octadecatetraenoic acid, gadoleic acid, eicosadienoic acid, arachidonic acid, eicosapentenoic acid, erucic acid, docosapentenoic acid and docosahexaenoic acid.

21. The emulsifier according to claim 20, wherein at least 50% by weight of said unsaturated fatty acid moieties are selected from oleic acid, linoleic acid and linolenic acid

22. The emulsifier according to claim 14 wherein said fatty acid moieties are derived from oils and/or fats of vegetable and/or animal origin.

23. The emulsifier according to claim 22, wherein said oil or fat is selected from the group consisting of almond oil, babassu oil, butter oil, chicken fat, cocoa butter, coconut oil, cotton seed oil, fish oil, hazelnut oil, illipe fat, kokorm, lard, linseed oil, maize oil, mango, menhaden oil, olive oil, palm kernel oil, palm oil, peanut oil, high erucic rapeseed oil, low erucic rapeseed oil, safflower oil, high oleic safflower oil, seal oil, shea fat, single cell algae oil, soybean oil, sunflower oil, high oleic sunflower oil, tall oil, tallow and a mixture of any of these oils and/or fats, preferably sunflower oil or high oleic sunflower oil.

24. The emulsifier according to claim 23, wherein the content of unsaturated fatty acid in said oil and/or fat has been reduced by partial and/or selective hydrogenation of unsaturated fatty acids.

25. The emulsifier according to claim 14, wherein said LACTEM has an iodine value above 6, preferably between 10 and 120, more preferably between 20 and 100, most preferably between 35 and 75.

26. The emulsifier according to claim 14, wherein said monoglycerides comprise from 40 to 99.5%, preferably from 60 to 99%, more preferably from 75 to 97% of said mono- and diglycerides.

27. The use of a composition comprising lactic acid esters of mono- and diglycerides of unsaturated fatty acid(s) as an emulsifier for improving the firmness, volume and/or texture of products produced with said emulsifier.

28. The use according to claim 27, wherein said emulsifier is used for improving the firmness of products whipped with said emulsifier.

29. The use according to claim 27, wherein said emulsifier is based on from 95 to 5% by weight unsaturated fatty acids and from 5 to 95% by weight saturated fatty acids.

30. The use according to claim 29, wherein at least 10%, preferably at least 20%, more preferably at least 30% of said fatty acids comprise unsaturated fatty acids.

31. The use according to claim 27, wherein 55 to 45% by weight of said fatty acids are unsaturated fatty acids.

32. The use according to claim 27, wherein said composition also contains non-esterified glycerides of fatty acids and comprises from 5 to 50%, preferably 10 to 30%, most preferably 13 to 25% by weight of lactic acid moieties, calculated as lactic acid on the total weight of said composition after saponification.

33. The use according to claim 27, wherein said composition is based on unsaturated fatty acid(s) selected from oleic acid, linoleic acid and linolenic acid.

34. The use according to claim 27, wherein said whipped product is a food product.

35. The use according to claim 27, wherein said food product is a fat containing food.

36. The use according to claim 27, wherein said food product is a protein containing food product.

37. The use according to claim 36, wherein said protein is selected from the group consisting of milk protein, caseinate, whey protein, soy protein, pea protein, oat protein, potato protein, cottonseed protein and mixtures thereof.

38. The use according to claim 34, wherein said food product is selected from a mousse, a whipped dairy cream, a decoration cream, a topping, a vegetable fat whipping cream (imitation dairy cream), an ice cream, a sour dairy product, a cake margarine, a cake mix or a whipped filling.

39. A food or non-food product, which contains an effective amount of an emulsifier comprising lactic acid esters of mono- and diglycerides of unsaturated fatty acid(s) for improving the firmness, volume and/or texture of said product.

40. The product according to claim 39, which is a whipped food product.

41. The product according to claim 39, wherein said unsaturated fatty acids comprise from 5 to 95%, preferably from 30 to 55% of the fatty acid moieties of said emulsifier.

42. The whipped product according to claim 41, which is a food product containing fat and/or protein.
